(19) United States
(12) Patent Application Publication
(10) Pub. No.: US 2022/0395162 A1
(43) Pub. Date: Dec. 15, 2022

(54) SYSTEM AND METHOD FOR REMOTELY MANEUVERING A MAGNETIC MINIATURE DEVICE

(71) Applicants: Eran OREN, Tel Aviv (IL); Costin IFRIM, Orange, CA (US); Michael SHPIGELMACHER, Los Angeles, CA (US); BIONAUT LABS LTD., Herzliya (IL)

(72) Inventors: Eran OREN, Tel Aviv (IL); Costin IFRIM, Orange, CA (US); Michael SHPIGELMACHER, Los Angeles, CA (US)

(73) Assignee: BIONAUT LABS LTD., Herzliya (IL)

(21) Appl. No.: 17/776,543

(22) PCT Filed: Nov. 16, 2020
(86) PCT No.: PCT/US20/60677

§ 371 Date: May 12, 2022

Related U.S. Application Data
(60) Provisional application No. 62/936,352, filed on Nov. 15, 2019.

(51) Int. Cl. A61B 1/00; A61B 1/04

(52) U.S. Cl. CPC A61B 1/00158; A61B 1/041

(57) ABSTRACT

A system configured to remotely maneuver a magnetic miniature device within a patient along a path conforming to a predetermined route is provided. The system comprises two coils, each configured to produce a magnetic field, and to be selectively pivoted about at least a first pivot axis within a first predetermined range of angles, a horizontal platform configured to support thereon the patient and to be disposed within the coils, and a controller configured to direct operation of the system. The predetermined range of angles constrains the system from maneuvering the miniature device along the route. The controller is configured to calculate a path comprising a plurality of segments, the path conforming to the route within a predetermined deviation. The controller is further configured to operate the coils within the predetermined range of angles to induce a magnetic field to maneuver the miniature device along the path.

### FIELD OF THE INVENTION

The presently disclosed subject matter relates to systems and miniature device configured to navigate within a patient to deliver a payload to a predetermined location therewithin, and in particular to such systems which use magnetic fields to direct operation of miniature devices within a patient.

### BACKGROUND

Remote control of medical devices moving inside the human body can be useful for a variety of purposes, including delivery of therapeutic payloads, diagnostics or surgical procedures. Such devices may include microscale or nanoscale robots, medical tools, “smart pills,” etc. Such devices may be able to move in the body either through self-propulsion or an external propulsion mechanism. Accurate location and tracking of such devices may be necessary to ensure their proper functioning at the right anatomical location, and more specifically accurate delivery of the therapeutic payloads and/or diagnostics substances.

### SUMMARY

According to an aspect of the presently disclosed subject matter, there is provided a system configured to remotely maneuver a magnetic miniature device within a patient along a path conforming to a predetermined route, the system comprising:
two coils, each configured to produce a magnetic field, and to be selectively pivoted about at least a first pivot axis within a first predetermined range of angles;
a horizontal platform configured to support thereon the patient and to be disposed within the coils; and
a controller configured to direct operation of the system;
   wherein the predetermined range of angles constrains the system from maneuvering the miniature device along the route, and wherein the controller is configured to calculate a path comprising a plurality of segments, the path conforming to the route within a predetermined deviation, the controller being further configured to operate the coils within the predetermined range of angles to induce a magnetic field to maneuver the miniature device along the path.

The coils, in their respective middle positions, may be disposed such that through-going coil axes thereof are parallel to one another.

The coils, in respective middle positions, may be disposed such that they are coaxial with one another.

The first pivot axis may be substantially vertical.

Each of the coils may be further configured to be selectively pivoted about a second pivot axis within a second predetermined range of angles.

The first and second pivot axes of each coil may be substantially perpendicular to one another.

The system may be configured to selectively move the platform within the coils.

The movement of the platform within the coils may comprise linear motion along a horizontal platform axis.

The movement of the platform within the coils may further comprise linear motion along a horizontal axis perpendicular to the platform axis. The horizontal axis may be parallel to the first pivot axis.

The movement of the platform within the coils may comprise pivoting about a horizontal axis.

Each of the coils may comprise an electromagnet.

The system may be configured such that electricity is applied to each of the electromagnets in a direction opposite to that of the other of the electromagnets.

The electromagnets may comprise a superconducting material.

Each of the coils may comprise a fixed magnet.

The predetermined range of angles may be less than about 120°. The predetermined range of angles may be less than about 100°. The predetermined range of angles may be less than about 90°.

The internal diameter of the coils may be less than about 75 cm. The internal diameter of the coils may be less than about 60 cm. The internal diameter of the coils may be less than about 50 cm.

According to another aspect of the presently disclosed subject matter, there is provided a method of operating a system as described above to maneuver a magnetic miniature device within a patient, the method comprising:
injecting a miniature device into the patient;
determining a route between a start point and an end point;
calculating a path, comprising one or more line segments along which the system is capable of maneuvering the miniature device and which conforms to the route;
determining how to operate components of the system to generate magnetic fields to maneuver the miniature device along the path; and
maneuvering the miniature device along the path.

The method may further comprise determining a maximum acceptable deviation from the route for one or more portions thereof.

The start point may be the present position of the miniature device, and the end point may be a target location.

The target location may be determined based on maneuvering instructions provided by a user.

The target location may be a predetermined location within the patient.

Determining how to operate components of the system may comprise selecting the strength of the magnetic field produced thereby.

Determining how to operate components of the system may comprise selecting a pivot angle of each of the coils about its respective first pivot axis.

Determining how to operate components of the system may comprise selecting a pivot angle of each of the coils about an axis perpendicular to its respective first pivot axis.

Determining how to operate components of the system may comprise selecting movement of the platform in one or more directions relative to coils.

The method may further comprise providing the system.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a perspective view of a system according to the presently disclosed subject matter;

FIG. 2 schematically illustrates a path of a miniature device, conforming to a route, as maneuvered by the system illustrated in FIG. 1; and

FIG. 3 illustrates a method of maneuvering a miniature device using the system illustrated in FIG. 1.

### DETAILED DESCRIPTION

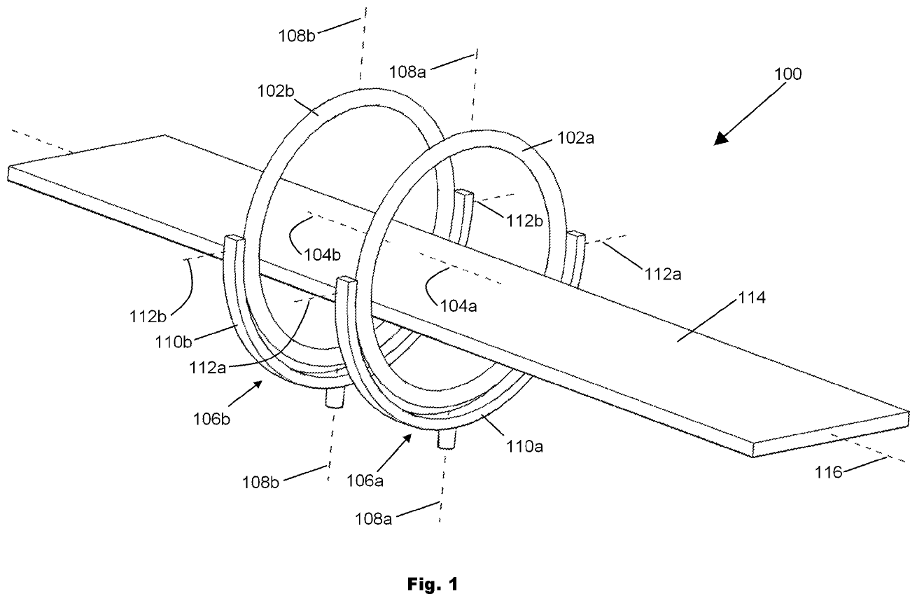

As illustrated in FIG. 1, there is provided a system, which is generally indicated at 100, for remotely maneuvering a magnetic device, in particular a miniature device, within a patient, for example to deliver one or more chemical compounds of medicinal, diagnostic, evaluative, and/or therapeutic relevance, one or more small molecules, biologics, cells, one or more radioisotopes, one or more vaccines, one or more mechanical devices, etc., to a predetermined location. The magnetic device may be microscale and/or nanoscale, and may be provided as described in any one or more of WO 19/213368, WO 19/213362, WO 19/213389, WO 20/014420, WO 20/092781, WO 20/092750, WO 18/204687, WO 18/222339, WO 18/222340, WO 19/212594, WO 19/005293, and PCT/US20/58964, the full contents of which are incorporated herein by reference.

The system 100 comprises first and second magnetic coils 102a, 102b. (Herein the specification and appended claims, similar elements designated by a base reference numeral and a trailing letter may be collectively designed by a generic form of the element name and/or collectively designated by the base reference numeral along; for example, the first and second coils may herein be collectively referred to as “coils” and/or collectively designated using reference numeral 102). According to some examples, each of the coils 102a, 102b comprises an electrical conductor disposed about a respective horizontal coil axis 104a, 104b, and is configured to produce a magnetic field, for example by having an electric current applied thereto, such as is well-known in the art.

It will be appreciated that herein the disclosure and claims, terms relating to orientation, such as “horizonal,” “vertical,” etc., and similar/related terms are used with reference to the orientation in illustrated in the accompanying drawings based on a typical usage of the system 100 and its constituent elements, unless indicated or otherwise clear from context, and are not to be construed as limiting. Similarly, terms relating to direction, such as “up,” “down,” and similar/related terms are used with reference to the orientation in illustrated in the accompanying drawings based on a typical usage of the system 100 and its constituent elements, unless indicated or otherwise clear from context, and are not to be construed as limiting.

According to some examples, the electrical conductor comprises a wire or other similar thin element wrapped a plurality of times around a rim made of a non-conductive material. (It will be appreciated that the term “non-conductive” and related terms as used herein the specification and appended claims includes materials having measurable but negligible conductivity.) According to some examples, the coils 102 are identical to one another, for example in size and/or number of wrappings of the conductive material. The system 100 may be configured to supply to same electric current to each of the coils and/or to supply different electric currents to the coils. The coils 102 may be electrically connected to one another, e.g., in series or in parallel, such that the same electrical current passes through both of them. The system 100 may be configured such that electrical current passes through the coils 102 in opposite and/or the same direction.

According to some examples, each of the coils 102 comprises a permanent magnet.

According to some examples, each of the coils comprises electromagnetic wires.

The coils 102 may each have a round shape, for example as illustrated in the accompanying figures, or may be formed having any other suitable shape, for example having a square shape, a hexagonal shape, etc. The coils 102 may each define a closed shape, for example as illustrated in the accompanying figures, or they may be open (not illustrated), for example defining a downwardly-facing C-shape, mutatis mutandis. According to some modifications, the coils 102 have a varying three-dimensional shape, i.e., their cross-sections taken at different planes perpendicular to their respective coil axes 104 are not uniform.

According to some examples, the coils 102 have an inner diameter which does not exceed about 50 cm. According to other examples, the coils 102 have an inner diameter which does not exceed about 60 cm. According to some examples, the coils 102 have an inner diameter which does not exceed about 75 cm.

Each of the coils 102a, 102b is mounted on a pivoting arrangement 106a, 106b, configured to selectively pivot its respective coil about a pivot axis 108a, 108b to a predetermined angular position. Similarly, each pivoting arrangement 106 may be further configured to rotate its respective coil 102 about its pivot axis 108 at a predetermined angular speed and/or speed profile (i.e., altering the angular speed according to a predetermined program). Accordingly, each pivot arrangement 106 comprises one or more suitable mechanisms to facilitate mechanical rotation thereof, including, but not limited to, one or more servo motors, one or more stepper motors, suitable gear trains, transmission systems, etc.

According to some examples, each of the pivoting arrangements 106 is configured to pivot its respective coil 102 within a predetermined range of angular positions, for example approximately ±45° from a middle position (total range of approximately 90°). According to some examples, the total range of angular positions is no more than about 100°. According to some examples, the total range of angular positions is no more than about 120°. According to some examples, the range of angular positions of the coils is restricted by a platform 114 (described below) passing therethrough.

Each of the pivoting arrangements 106 may further comprise a sensing system (not illustrated) configured to detect the angular position of its respective coil 102, for example using any suitable arrangement, such as is well-known in the art.

According to some examples, each of the pivot axes 108 is disposed at a perpendicular orientation relative to the coil axis 104 of its respective coil 102. According to some examples, the coils 102 are disposed such that when they are each in their respective middle position, their coil axes 104 are parallel to one another, for example being coincident with one another.

Each of the pivoting arrangements 106a, 106b may comprise a gimbal 110a, 110b carrying its respective coil 102a, 102, and configured to selectively pivot it about a gimbal axis 112a, 112b to a predetermined angular position. Similarly, each gimbal 110 may be further configured to rotate its respective coil 102 about its gimbal axis 112 at a predetermined angular speed and/or speed profile. Accordingly, each gimbal 110 comprises one or more suitable mechanisms to facilitate mechanical rotation thereof, including, but not limited to, one or more servo motors, one or more stepper motors, suitable gear trains, transmissions, etc.

According to some examples, each of the gimbals 110 is configured to pivot its respective coil 102 within a predetermined range of angular positions, for example approximately ±45° from a middle position (total range of approximately 90°).

Each of the gimbals 110 may further comprise a sensing system (not illustrated) configured to detect the angular position of its respective coil 102, for example using any suitable arrangement, such as is well-known in the art. Moreover, it will be appreciated that the system 100 may comprise a single sensing system configured to detect the angular position of each coil 102 about its respective pivot axis 108 and gimbal axis 112, for example as is well-known in the art.

According to some modifications, one or both of the pivoting arrangements 106 is configured to selectively move vertically, i.e., along its pivot axis 108. Each pivoting arrangement 106 may be configured to move independently of the other, or both may be configured to move in unison.

According to some modifications, the system 100 does not comprise the pivoting arrangements 106, i.e., it may comprise the gimbals 110 as independent units. Accordingly, the system 100 may be configured to pivot each of the coils 102 about its respective gimbal axis 110 only.

The system 100 may further comprise a platform 114, configured to support thereon the patient, e.g., in a horizontal position, such as a supine or a prone position. The platform 114 may be made of a material which does not, or only negligibly, interfere with or react to the magnetic field produced by the coils 102.

According to some examples, each of the coils 102 has a diameter which is only slightly larger than the width of the platform 114, for example limiting the range of pivoting about its pivot axis 108 to about ±45°, for example as alluded to above.

The system 100 may be configured to selectively move the platform 114 along a horizontal platform axis 116, e.g., which is mutually perpendicular to the pivot and gimbal axes 108, 110. According to some examples, the platform axis 116 is parallel to the coil axes 104 when the coils 102 are in their respective middle positions.

The system 100 may be further configured to selectively move the platform 114 in a vertical direction, i.e., parallel to the pivot axis 108, and/or to selectively tilt the platform 114 about one or more horizontal axes, e.g., parallel to the gimbal axis 112, parallel to the platform axis 116, etc.

The system 100 may comprise a platform drive mechanism (not illustrated), configured to facilitate movement of the platform 114. The platform drive mechanism may comprise one or more servo motors, one or more stepper motors, suitable gear trains, transmission systems, and/or any other elements suitable to effect the desired movement of the platform 114.

The system 100 may further comprise a controller (not illustrated) configured to direct operation of the components thereof. It will be appreciated that while herein the specification and claims, the term “controller” is used with reference to a single element, it may comprise a combination of elements, which may or may not be in physical proximity to one another, without departing from the scope of the presently disclosed subject matter, mutatis mutandis. In addition, disclosure herein (including recitation in the appended claims) of a controller carrying out, being configured to carry out, or other similar language, implicitly includes other elements of the system 100 carrying out, being configured to carry out, etc., those functions, without departing from the scope of the presently disclosed subject matter, mutatis mutandis.

The system 100 may further comprise a power source (not illustrated), configured to provide electrical power to the components thereof. The power source may comprise, but is not limited to, an energy storage device, a rectifier, a linear regulator, an inverter, a transformer, and/or any other suitable device configured to provide required electrical power from storage or from an external source.

The system 100 may further comprise safety mechanisms, for example to ensure that temperatures and magnetic fields induced by the system remain within safe levels. It may further comprise and/or be configured to interface with imaging systems, including, but not limited to, systems using x-rays, computed tomography, ultrasound, positron emission tomography, single-photon emission computed tomography, etc.

The system 100 may be characterized by the number of degrees of freedom it may operate with. Each additional motion of the patient relative to the coils 102 which the system 100 is capable of effecting may be associated with an additional degree of freedom. For example, providing two coils 102, each configured to pivot along a pivot axis 108 and a gimbal axis 112 may be associated with two degrees of freedom, and providing a platform 114 which is configured to move in three orthogonal directions (parallel to each of the pivot, gimbal, and platform axes 108, 112, 116) may be associated with an additional three degrees of freedom.

In use, the system 100 may be used to maneuver the magnetic miniature device by selectively varying the magnetic field produced thereby. The miniature device is injected into the patient, e.g., into the cerebrospinal fluid (CSF), e.g., in the spinal cord, for example for being maneuvered toward the brain. The patient is positioned on the platform 114, and within the coils 102. The system 100, for example based on imaging, e.g., X-ray images, may determine a route between a start point and an end point. According to some examples, a user manually provides maneuvering instructions to the system 100, for example providing input defining a route while monitoring the miniature device within the patient.

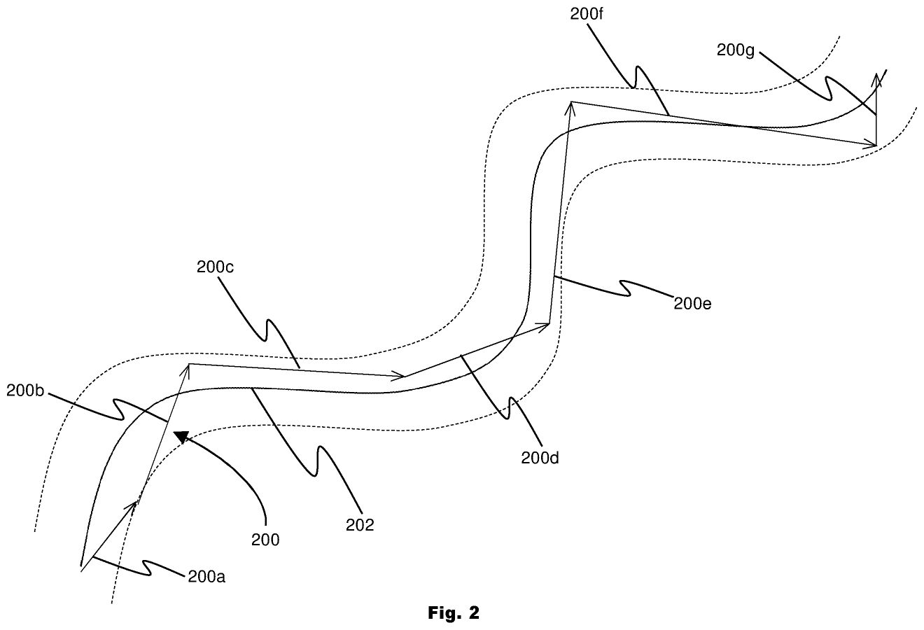

The magnetic field may be varied, inter alia, by pivoting the coils 102 about their respective pivot and/or gimbal axes 112. As illustrated in FIG. 2, as the range of pivoting of the coils 102 is limited by the presence of the platform 114 therethrough, the system 100 is configured to direct the miniature device along a zigzag path 200, e.g., comprising a plurality of segments 200a-g, e.g., straight line segments, the path closely conforming to the desired route 202. This may be accomplished, for example, by strategically alternating the directions of the magnetic forces acting on the miniature device.

For the sake of clarity, herein the specification and appended claims, the term “route” is used to indicate the target course of the miniature device, for example as determined by the system 100 and/or as input by a user, and the term “path” is used to indicate the actual course along which the system 100 maneuvers the miniature device, for example comprising a plurality of straight line segments, as described above with reference to and as illustrated in FIG. 2.

Accordingly, according to some examples the system 100 may be configured to determine a route, for example as mentioned above, and then to calculate a path of line segments along which it is capable of maneuvering the miniature device, within the physical constraints of the coils, and which conforms to the route. According to other examples, a user may navigate the miniature device by providing instructions to the system 100, for example as mentioned above, e.g., in real time, to define the route along which the miniature device should travel; the system 100 is configured to calculate a path comprising line segments along which it is capable, within the physical constraints of the coils, of maneuvering the miniature device, conforming to the route.

It will be appreciated that while the system 100 must compensate for its physical constraints by maneuvering the miniature device along a path which approximates a desired route, this approximation is often an acceptable one. Moreover, these constraints are a consequence of the system 100 providing fewer degrees of freedom than would be necessary for the path of the miniature device to fully conform to the determined route; however, a system characterized by such constraints and configured to determine a zigzag path described above may be provided smaller and use less electricity than a system having the necessary degrees of freedom to maneuver the miniature device along a desired route without deviating therefrom at all. For example, a system comprising coils which are capable of pivoting through 360° would necessarily require much larger coils; as the magnetic field is stronger at the closer range to the miniature device, it requires much less power to provide the same magnetic force thereto.

It will be further appreciated that the system 100 may be capable of maneuvering the miniature device, inter alia, along some non-linear portions of routes, for example based on the position of the patient relative to the coils 102. In such cases, for such portions, the system 100 may maneuver the miniature device along a path which coincides with the route.

The system 100 may be configured to calculate the path based on any suitable method. For example, the system 100 may be provided with and/or determine a maximum acceptable deviation σ from the path for each point therealong, e.g., based on physiological constraints. For example, thde value of σ may be smaller in highly sensitive areas of the brain than they are in portions of the spinal cord which define a relatively wide area through which it is safe to maneuver the miniature device. Accordingly, the system 100 may be configured to calculate a path along which it is capable of maneuvering the miniature device, and comprising line segments whose deviation from the route do not exceed σ.

The system 100 may be further capable of determining how to operate its components (e.g., what angles to pivot each of the coils 102 along its respective pivot and/or gimbal axes 108, 112, what strengths the magnetic fields induced by the coils should be, how the platform 114 should be moved, etc.) in order to maneuver the miniature device along the path and/or along a path which acceptably conforms (i.e., within σ) to the route. This determination may made based on any suitable method.

According to some examples, the system 100 is configured to determine how to operate its components in order to generate the necessary magnetic fields by calculating the spatial magnetic field distribution around the coils 102 in different orientations thereof, and in different positions of the miniature device relative thereto, in order to facilitate arriving at this determination. According to some examples, the system 100 calculates this using the Biot-Savart law:

$$B ( r ) = \frac{{μ}_{0}}{4 π} {\int }_{C} \frac{Id ℓ \times {\hat{r}}^{′}}{{\vert {r}^{′} \vert}^{2}}$$

in which B(r) is the magnetic field at position r, dl is a vector along path C whose magnitude is the length of a differential element of the wire through which current I flows in the direction of conventional current, l is a point on path C, r′=r−l is the full displacement vector from the wire element (dl) to the point at point l to the point at which the field is being computed (r), \hat{r}′ is the unit vector of r′, and μ₀ is the magnetic constant. The kinematics may be calculated over short distances using the Lorentz equation:

F=qE+qv×B

in which F is the force experienced by a particle having a charge q with a velocity v in electric field E and magnetic field B.

The Biot-Savart law and/or Lorentz equations may be applied with any suitably reasonable approximations and/or analytical forms of the spatial magnetic field distribution, for example as is well-known in the art. Alternatively or in addition, the system may be configured to perform a finite element analysis to calculate the magnetic field.

According to other examples, the system 100 is configured to determine how to operate its components in order to generate the necessary magnetic fields using a trial-and-error approach. For example, the system 100 may be configured to monitor the reaction of the miniature device when applying a magnetic field thereto. The monitoring may be visual, for example using image-recognition software on x-ray images, and/or the monitoring may comprise obtaining feedback from the miniature device by inducing a magnetic pulse. Such an approach may be used in real time, i.e., modifying operational parameters of the system 100 based on how closely the miniature device confirmed to the route and/or path, and/or the reactions of the miniature device to a set of operational parameters may be used to train an artificial intelligence (i.e., machine learning) model to train the system 100 to utilize its components to predictably control the miniature device by varying the operational parameters of its components. Any suitable machine learning algorithm may be used, for example using an artificial neural network applying a Deep Q-learning algorithm. Such machine learning approaches may be performed in-vitro, i.e., in a simulated environment not using a live patient, for example in a human or non-human cadaver, in a model of a patient, etc., and/or in-vivo.

A trial-and-error approach may include inducing a magnetic field, and varying it when the miniature device deviates from the path by more than σ in any direction. It may further include selectively reversing the magnetic in order to maneuver the miniature robot along a path in a reverse direction to that immediately preceding it.

According to some examples, the above approaches may be combined, i.e., a computational approach such as described above may be used to calculate initial conditions, predicted responses of the miniature devices, etc., and this information is used to refine a trial-and-error approach (performed in real time and/or as part of a machine learning algorithm).

While an example of maneuvering a single miniature device has been described, it will be appreciated that the system 100 may be used to maneuver two or more miniature devices within a patient, for example simultaneously and/or sequentially. The miniature devices may be free and/or tethered to one another and/or to an external element, such as a catheter.

It will be appreciated that while an example of the system 100 having two coils 102 is described herein, this is by way of example only, and the presently disclosed subject matter is not limited thereto. The system 100 may be provided with three, four, or any other suitable number of coils 102 and accompanying elements (pivoting arrangements 106, gimbals 110, etc.) without departing from the scope of the presently disclosed subject matter, mutatis mutandis.

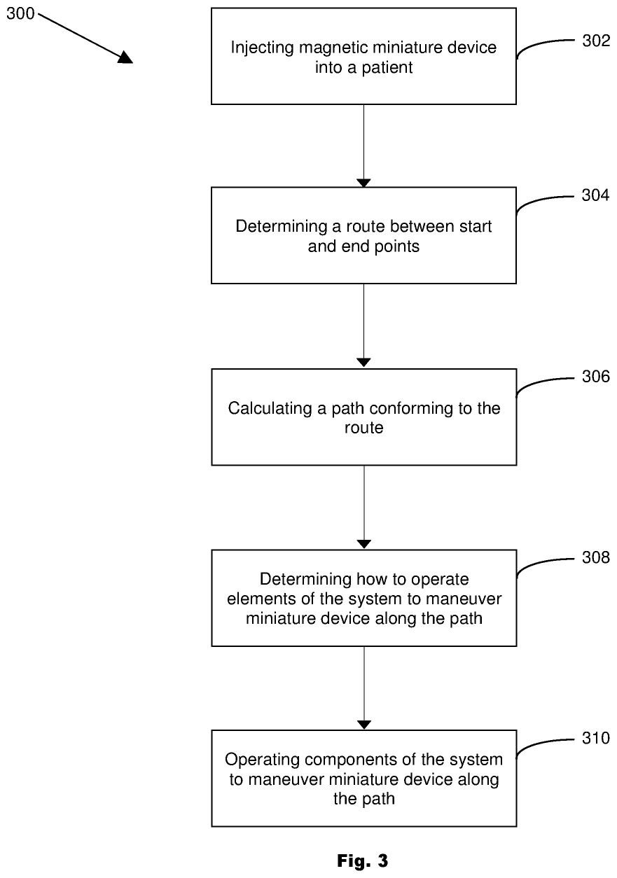

As illustrated in FIG. 3, a method, generally indicated at 300, may be configured. In a first step 302, a magnetic miniature device is injected into a patient. In a second step 304, the system 100 determines a route between a start point and an end point. The route may be determined based on the present position of the miniature device and a target position, and/or it may be determined based on maneuvering instructions provided by a user, e.g., in real time. In a third step 306, the system 100, e.g., the controller thereof, calculates a path, comprising one or more line segments along which the system is capable of maneuvering the miniature device and which conforms to the route, i.e., does not deviate therefrom more than a predefined about σ. In step 308, the system 100 determines how to operate its components in order to generate the necessary magnetic fields to maneuver the miniature device along the path. In step 310, the system operates its components accordingly, thereby maneuvering the miniature device along the path.

It will be appreciated that while the method 300 is described above as having first, second, third, etc., steps, this is not to be construed as limiting; the steps of the method so described may be carried out in any suitable order, including, but not limited to, performing portions of one or more single steps out of the order implied herein, without departing from the scope of the presently disclosed subject matter, mutatis mutandis. Moreover, it will be appreciated that one or more of the steps of the method 300 and/or portions thereof may be performed iteratively, including, but not limited to, monitoring the miniature device and revisiting previously performed steps, mutatis mutandis.

It will be recognized that examples, embodiments, modifications, options, etc., described herein are to be construed as inclusive and non-limiting, i.e., two or more examples, etc., described separately herein are not to be construed as being mutually exclusive of one another or in any other way limiting, unless such is explicitly stated and/or is otherwise clear.

Those skilled in the art to which this invention pertains will readily appreciate that numerous changes, variations, and modifications can be made without departing from the scope of the presently disclosed subject matter, mutatis mutandis.

1. A system configured to remotely maneuver a magnetic miniature device within a patient along a path conforming to a predetermined route, the system comprising:
two coils, each configured to produce a magnetic field, and to be selectively pivoted about at least a first pivot axis within a first predetermined range of angles;
a horizontal platform configured to support thereon the patient and to be disposed within said coils; and
a controller configured to direct operation of the system;
wherein said predetermined range of angles constrains the system from maneuvering the miniature device along the route;
wherein said controller is configured to calculate a path comprising a plurality of segments, the path conforming to said route within a predetermined deviation, the controller being further configured to operate said coils within said predetermined range of angles to induce a magnetic field to maneuver the miniature device along said path.

2. The system according to claim 1, said coils, in respective middle positions, being disposed such that through-going coil axes thereof are parallel to one another.

3. The system according to claim 1, said coils, in respective middle positions, being disposed such that they are coaxial with one another.

4. The system according to any one of the preceding claims, wherein said first pivot axis is substantially vertical.

5. The system according to any one of the preceding claims, wherein each of the coils is further configured to be selectively pivoted about a second pivot axis within a second predetermined range of angles.

6. The system according to claim 5, wherein said first and second pivot axes of each coil are substantially perpendicular to one another.

7. The system according to any one of the preceding claims, configured to selectively move said platform within said coils.

8. The system according to claim 7, wherein the movement of the platform within said coils comprises linear motion along a horizontal platform axis.

9. The system according to claim 8, wherein the movement of the platform within said coils further comprises linear motion along a horizontal axis perpendicular to said platform axis.

10. The system according to claim 9, said horizontal axis being parallel to said first pivot axis.

11. The system according to any one of claims 7 through 10, wherein the movement of the platform within said coils comprises pivoting about a horizontal axis.

12. The system according to any one of the preceding claims, wherein each of said coils comprises an electromagnet.

13. The system according to claim 12, configured such that electricity is applied to each of said electromagnets in a direction opposite to that of the other of the electromagnets.

14. The system according to any one of claims 12 and 13, wherein said electromagnets comprise a superconducting material.

15. The system according to any one of the preceding claims, wherein said coils each comprises a fixed magnet.

16. The system according to any one of the preceding claims, wherein said predetermined range of angles is no more than about 120°.

17. The system according to claim 16, wherein said predetermined range of angles is no more than about 100°.

18. The system according to claim 17, wherein said predetermined range of angles is no more than about 90°.

19. The system according to any one of the preceding claims, wherein the internal diameter of the coils does not exceed about 75 cm.

20. The system according to claim 19, wherein the internal diameter of the coils does not exceed about 60 cm.

21. The system according to claim 19, wherein the internal diameter of the coils does not exceed about 50 cm.

22. A method of operating a system according to any one of the preceding claims to maneuver a magnetic miniature device within a patient, the method comprising:
injecting a miniature device into the patient;
determining a route between a start point and an end point;
calculating a path, comprising one or more line segments along which the system is capable of maneuvering the miniature device and which conforms to the route;
determining how to operate components of the system to generate magnetic fields to maneuver the miniature device along the path; and
maneuvering the miniature device along the path.

23. The method according to claim 22, further comprising determining a maximum acceptable deviation from the route for one or more portions thereof.

24. The method according to any one of claims 22 and 23, wherein the start point is the present position of the miniature device, and the end point is a target location.

25. The method according to claim 24, wherein said target location is determined based on maneuvering instructions provided by a user.

26. The method according to claim 24, wherein said target location is a predetermined location within the patient.

27. The method according to any one of claims 22 through 26, wherein determining how to operate components of the system comprises selecting the strength of the magnetic field produced thereby.

28. The method according to any one of claims 22 through 27, wherein determining how to operate components of the system comprises selecting a pivot angle of each of said coils about its respective first pivot axis.

29. The method according to any one of claims 22 through 28, wherein determining how to operate components of the system comprises selecting a pivot angle of each of said coils about an axis perpendicular to its respective first pivot axis.

30. The method according to any one of claims 22 through 29, wherein determining how to operate components of the system comprises selecting movement of the platform in one or more directions relative to coils.
